(19) United States
(12) Patent Application Publication
(10) Pub. No.: US 2005/0208131 A1
(43) Pub. Date: Sep. 22, 2005

(54) Method of preparing pharmaceutical dosage forms containing multiple active ingredients

(76) Inventors: Gisele Veilleux, Laval (CA); Victor Shulman, Thornhill (CA); Eric Gervais, Laval (CA)

Correspondence Address: FULBRIGHT & JAWORSKI L.L.P., 600 CONGRESS AVE., SUITE 2400, AUSTIN, TX, 78701, US

(21) Appl. No.: 10/670,907

(22) Filed: Sep. 25, 2003

(30) Foreign Application Priority Data
Oct. 4, 2002 (CA) 2,406,592

(51) Int. Cl. A61K 9/20; B27N 3/00

(52) U.S. Cl. 424/465; 264/109

(57) ABSTRACT

A method for the preparation of pharmaceutical dosage forms comprising multiple powdered active ingredients, in a preferred embodiment, said method comprising the steps of mixing said active ingredients and at least one chosen excipient so as to obtain a powdered mixture; compacting said powdered mixture in a roller-compactor apparatus to obtain a compacted product; breaking and sieving said compacted product to a chosen mesh size to obtain similar sized granules; preferably dry mixing said granules with at least on chosen excipient so as to obtain a granular mixture; forming said granular mixture into unitary dosage forms.

### FIELD OF THE INVENTION

The present invention relates to a method of preparing pharmaceutical dosage forms containing multiple active ingredients. More specifically, the present invention is concerned with alleviating active ingredient losses during manufacturing and ensuring content uniformity of dosage forms.

### BACKGROUND OF THE INVENTION

A number of pharmaceutical dosage forms comprise multiple active ingredients. One example is the anti-nauseant medicament prescribed during pregnancy currently sold in Canada under the trademark Diclectin®.

Diclectin® is a medicament containing a synergistic duo of active ingredients, namely Doxylamine Succinate and Pyridoxine HCl. In the case of Diclectin®, the approved label of the product calls for the duo of active ingredients to be present in exactly equal amounts of 10 mg. These active ingredients are obtained in the form of powders having different granular sizes which makes it very difficult to uniformly mix them in a dry state along with required excipients. Such phenomenon is generally caused by particle segregation during mixing. This poses a content uniformity challenge during manufacturing of final dosage forms.

An added challenge to content uniformity is the loss of the active ingredient Pyridoxine HCl during manufacturing of Diclectin®. Pyridoxine HCl is generally provided as a crystalline powder having a mean particle diameter of about 60 microns. In contrast, Doxylamine Succinate is composed of rod shaped particles having a mean particle diameter of about 200 microns. It has been observed that due to their small size and possible electrostatic charge, Pyridoxine HCl particles tend to easily adhere to manufacturing vessels and other processing or storage equipment. Thus, when processing both active ingredients through the same equipment, more Pyridoxine HCl is lost than Doxylamine Succinate. To compensate for this effect, operators have commonly used a 8-10 weight percent overage of Pyridoxine HCl in comparison to Doxylamine Succinate. However, the result of such method is somewhat irregular and quality controls still reject many lots.

In general terms, whenever preparing multi-ingredient medicaments, it is important that manufacturing methods allow for the final content of each dosage form to follow rather exactly the contents announced on the label. This is indeed a legal and regulatory requirement in most countries of the world.

Thus, there is a need for a method of manufacturing Diclectin® or other similar powderous multi-ingredient medicaments which alleviate ingredient losses during manufacturing and provides superior content uniformity results when compared to known methods.

### OBJECTS OF THE INVENTION

Objects of the present invention are therefore to provide an improved method of preparing pharmaceutical dosage forms containing multiple active ingredients so as to ensure active ingredient content uniformity and to alleviate active ingredient losses during manufacturing.

### SUMMARY OF THE INVENTION

More specifically, in accordance with the present invention, there is provided a method for the preparation of pharmaceutical dosage forms comprising multiple powdered active ingredients, said method comprising the steps of:
(a) mixing said active ingredients and at least one chosen excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) forming said granules into unitary dosage forms.

In another aspect, the method may comprise the steps of:
(a) mixing said active ingredients and at least one chosen excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one chosen excipient so as to obtain a granular mixture;
(e) forming said granular mixture into unitary dosage forms.

In yet another aspect, the method may comprise the steps of:
(a) mixing said active ingredients so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one chosen excipient so as to obtain a granular mixture;
(e) forming said granular mixture into unitary dosage forms.

In yet another aspect, the method may comprise the steps of:
(a) mixing at least one of said active ingredients and at least one excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one other active ingredient so as to obtain a granular mixture;
(e) forming said granular mixture into unitary dosage forms.

In yet another aspect, the method may comprise the steps of:
(a) mixing at least one of said active ingredients and at least one excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one other active ingredient and at least one other excipient so as to obtain a granular mixture;
(e) forming said granular mixture into unitary dosage forms.

Other aspects, objects, advantages and features of the present invention will become more apparent upon reading of the following non-restrictive description of preferred embodiments thereof, given by way of example only with reference to the accompanying drawing.

### BRIEF DESCRIPTION OF THE DRAWING

In the appended drawing:

FIG. 1 is a flowchart of a preferred embodiment of the manufacturing method steps of the present invention.

### DESCRIPTION OF THE PREFERRED EMBODIMENT

When used herein, the term “active ingredient” refers to a therapeutically active substance. “Therapeutically active substance” is to be understood to encompass vitamins or nutritional supplements.

When used herein, the term “medicament” refers to a pharmaceutical dosage form comprising one or more active ingredients and one or more excipients and optionally one or more coatings.

The prior art method of manufacturing Diclectin®, a medicament containing a synergistic duo of active ingredients consisted of dry mixing the active ingredients along with excipients; the mixed powder was then compacted into a tablet shape and appropriately coated.

It has now been found against expectations that the use of a roller compactor alleviates active ingredient losses during manufacturing. As an added benefit, content uniformity in terms of active ingredients is vastly improved because the particle size of active ingredients may now be standardized thereby avoiding poor mixing of active ingredients or losses due to fines which adhere to processing equipment or which do not flow properly. Indeed, roller compaction allows fine powders to be augmented to larger size particles that are less prone to cause ingredient losses during processing.

In the preferred embodiment wherein at least two active ingredients are roller compacted together, additional benefits are apparent. In such case, the powdered active ingredients are augmented in particle size in a physically combined entity of the active ingredients. This entity now resists particle segregation upon mixing and allows for improved mixing of the two active ingredients.

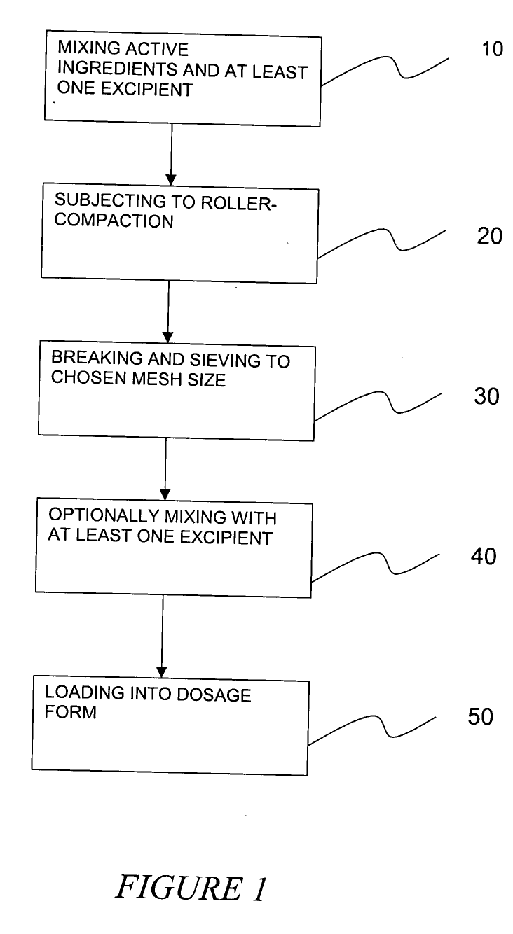

Referring to FIG. 1, there is shown a schematic flowchart of a preferred embodiment of the process of the present invention. In general terms, in a first step 10, the active ingredients are mixed, preferably dry mixed, with at least one chosen excipient to obtain a powdered mixture. The next step 20 is to submit the powdered mixture to roller compaction to obtain a compacted product. In step 30 the compacted product is broken and sieve to a chosen mesh size. Step 40 is an optional step wherein the resulting granulate of step 30 is mixed with one or more excipients and or other active ingredients. In step 50, the resulting product from step 40 is loaded into a final dosage form such as a tablet shape obtained by compression.

A roller compactor is essentially a piece of equipment capable of compacting a powdered substance into a friable compacted product. The Chilsonator® sold by Fitzpatrick Company of Elmhurst, Ill., USA are examples of such equipment. Roller compactors are commonly provided with a hopper into which the powdered substance is loaded. Counter-rotating rollers force the powdered substance between compaction rollers below or to the side of the hopper discharge. The shape of the resulting compacted product, its harness and density are essentially dictated by the relative distance and speed of the rollers, the speed of the hopper infeed and the compaction properties of the materials being compacted.

When using a roller compactor to compact an initial blend of powdered ingredients, the resulting compacted product may be broken and sieved to a chosen mesh size to achieve a specified granule size distribution. To this end, a breaking rotor or wheel and a vibrating mesh screen are commonly used. Fines are usually discarded or recycled back into the hopper. The resulting granulate may be further blended to ensure content uniformity of initial ingredients throughout the resulting granulate.

In essence, the compaction process removes entrapped air from interstices of the initial substance and forms denser granules when broken. Also, fine powders having poor flow characteristics and subject to electrostatic charge causing unwanted adhere to processing or storage equipment, once subjected to roller compaction, are upgraded in size to larger granules which are less prone to cause ingredient losses during processing or storage.

Furthermore, since the resulting granulate is of essentially uniform size distribution, the problem of size difference of the initial powdered ingredients is addressed. For example, the ingredient Pyridoxine HCl and Doxylamine Succinate are no longer of different mean particle diameter and are of a mean particle diameter large enough to prevent excessive loss of Pyridoxine HCl during processing.

Example 1 below is a demonstration of active ingredient loss using a prior art manufacturing method. Example 2 that follows example 1 is a demonstration that such active ingredient loss is negated when practicing the method of the present invention.

### EXAMPLE 1

### Prior Art

Active ingredients, namely Pyridoxine HCl and Doxylamine Succinate were blended with exact quantities of excipients. Five samples of 3 grams were placed into small polyethylene bags and shaken. This mimics the prior art method of placing a final blend of active ingredients and excipients into polyethylene lined drums prior to emptying said drums into the hopper of a tablet compression machine. After being placed in the small polyethylene bags, the samples were removed and analyzed for content of active ingredients. The results are shown in Table I below:

TABLE I
Content analysis compared to initial quantity being 100% wt of
each of Pyridoxine HCI and Doxylamine Succinate.

| | PYRIDOXINE | DOXYLAMINE |
|---|---|---|
| SAMPLE No. | HCL IN WT % | SUCCINATE IN WT % |
| 1 | 76.6 | 101.3 |
| 2 | 77.6 | 104.1 |
| 3 | 85.9 | 101.4 |
| 4 | 85.3 | 101.4 |
| 5 | 87.1 | 101.6 |
| Average loss | 17.5% | Nil |

Note:
values above 100% are attributable to the detection limit of the analysis apparatus.

This example clearly shows how Pyridoxine HCl is prone to loss during processing and storage. Example 2 below shows how this problem is avoided by the method of the present invention.

### EXAMPLE 2

The active ingredients, namely Pyridoxine HCl and Doxylamine Succinate were blended with exact quantities of excipients as in Example 1. However, this time the blend was processed using a Chilsonator® Roller compactor to form compacted products that were then crushed and screened to 16 mesh. A series of six 3 grams samples were collected. Two of the samples were directly analyzed for active ingredient content. The four remaining samples were placed in small polyethylene bags and shaken as in Example 1. The samples were then removed from the bags and analyzed for active ingredient content.

The results are shown in Table II below:

TABLE II
Content analysis compared to control quantity being about
68.8 mg of Pyridoxine HCI per gram of mixture and about 67.5 mg of
Doxylamine Succinate per gram of mixture.

| | | DOXYLAMINE |
|---|---|---|
| SAMPLE No. | PYRIDOXINE HCL | SUCCINATE |
| 1 (control) | 68.6 mg/g | 67.9 mg/g |
| 2 (control) | 68.9 mg/g | 67.1 mg/g |
| Average of 1 (control) | 68.8 mg/g or 100 wt % | 67.5 mg/g or 100 wt % |
| and 2 (control) | | |
| 3 | 95.1 wt % vs. control | 99.6 wt % vs. control |
| 4 | 95.5 wt % vs. control | 100.6 wt % vs. control |
| 5 | 97.1 wt % vs. control | 100.4 wt % vs. control |
| 6 | 96.5 wt % vs. control | 99.4 wt % vs. control |
| Average loss | 3.9 wt % vs. control | Nil |

Note:
values above 100% are attributable to the detection limit of the analysis apparatus.

These results demonstrate that by using the manufacturing method of the present invention, the average loss of Pyridoxine HCl was dramatically lowered when compared to the prior art method.

It is also to be understood that the method of the present invention can also involve the step of blending the granules resulting from roller compaction to further increase content uniformity of the granular blend. This is done prior to compression into tablet shape or prior to placing the granules in some other suitable dosage form.

It is also to be understood that the method of the present invention can involve mixing the active ingredients alone, i.e. without excipients, and submitting the active ingredients to roller compaction prior to blending the compacted granules with at least one excipient.

It is also to be understood that the method of the present invention can involve mixing a single active ingredient (usually the smaller sized active ingredient) with at least one excipient and submitting the mixture to roller compaction prior to blending the compacted granules with at least one other active ingredient and perhaps other excipients.

It is also to be understood that all mixing steps can be accomplished as sequential mixing of various ingredients with or without intervening sieving or pre-blending steps. The term “mixing” is used in its broad sense of creating a mixture regardless of the exact processing steps used to obtain this mixture.

When compressed into tablet shape as for an oral or sublingual dosage form, the tablet can be sealed or otherwise coated such as with an enteric coating. The exact coating will of course depend on the intended release site and release rate of the active ingredients once the tablet is ingested.

Although the present invention has been described hereinabove by way of preferred embodiments thereof, it can be modified, without departing from the spirit and nature of the subject invention as defined in the appended claims.

1. A method for the preparation of pharmaceutical dosage forms comprising multiple powdered active ingredients, said method comprising:
(a) mixing said active ingredients and at lease one chosen excipient so as to obtain a powdered mixture;
(b) compacting said powdered mixture to obtain a compacted product;
(c) breaking and sieving said compacted product to a chosen mesh size to obtain similar sized granules; and
(d) forming said granules into unitary dosage forms.

2. A method for the preparation of pharmaceutical dosage forms comprising multiple powdered active ingredients, said method comprising:
(a) mixing said active ingredients and at lease one chosen excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one chosen excipient so as to obtain a granular mixture; and
(e) forming said granular mixture into unitary dosage forms.

3. A method for the preparation of pharmaceutical dosage forms comprising multiple powdered active ingredients, said method comprising:
(a) mixing said active ingredients so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one chosen excipient so as to obtain a granular mixture; and
(e) forming said granular mixture into unitary dosage forms.

4. A method for the preparation of pharmaceutical dosage forms comprising multiple powdered active ingredients, said method comprising the steps of.
(a) mixing at least one of said active ingredients and at least one excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one other active ingredient so as to obtain a granular mixture; and
(e) forming said granular mixture into unitary dosage forms.

5. A method for the preparation of pharmaceutical dosage forms comprising multiple powdered active ingredients, said method comprising:
(a) mixing at least one of said active ingredients and at least one excipient so as to obtain a powdered mixture;
(b) compacting said powdered mixture in a roller compactor apparatus to obtain a compacted product;
(c) breaking and sieving said compacted product to a chosen mesh size to obtain similar sized granules;
(d) mixing said granules with at least one other active ingredient and at least one other excipient so as to obtain a granular mixture; and
(e) forming said granular mixture into unitary dosage forms.

6. The method of claim 1 wherein the step of forming said granular mixture into unitary dosage forms comprises compressing said granular mixture into a tablet shape.

7. The method of claim 6 wherein the tablet shape is provided with a coating.

8. The method of claim 7 wherein said coating is an enteric coating.

9. The method of claim 1 wherein the step of forming said granular mixture into unitary dosage forms comprises loading said granular mixture into an open capsule and thereafter closing said capsule.

10. The method of claim 1 wherein the active ingredients comprise Pyridoxine HCl and Doxylamine Succinate.

11. The method of claim 1 wherein the active ingredients comprise equal parts of Pyridoxine HCl and Doxylamine Succinate.

12. The method of claim 1 wherein the active ingredients consist of equal parts of Pyridoxine HCL and Doxylamine Succinate.
